(19) United States
(12) Patent Application Publication
(10) Pub. No.: US 2022/0068466 A1
(43) Pub. Date: Mar. 3, 2022

(54) MEDICAL DATA FILE PROCESSING APPARATUS, AND MEDICAL DATA FILE PROCESSING METHOD

(71) Applicant: CANON MEDICAL SYSTEMS CORPORATION, Otawara-shi (JP)

(72) Inventors: Yohei KAMINAGA, Otawara (JP); Kouji OTA, Nasushiobara (JP); Hidetoshi ISHIGAMI, Otawara (JP); Koji TAKEI, Nasushiobara (JP); Daisuke SUZUKI, Otawara (JP); Yoshifumi YAMAGATA, Sakura (JP)

(73) Assignee: CANON MEDICAL SYSTEMS CORPORATION, Otawara-shi (JP)

(21) Appl. No.: 17/462,511

(22) Filed: Aug. 31, 2021

(30) Foreign Application Priority Data
Sep. 1, 2020 (JP) 2020-147030

(51) Int. Cl. G16H 30/20; G16H 50/20; G16H 10/60; G06F 21/62; G16H 50/70; G06K 9/62

(52) U.S. Cl. CPC G16H 30/20; G16H 50/20; G16H 10/60; G16H 30/40; G16H 50/70; G06K 9/6267; G06F 21/6254

(57) ABSTRACT

The medical data file processing apparatus according to the present embodiment includes processing circuitry. The processing circuitry is configured to acquire an image-interpretation report. The processing circuitry is configured to classify a medical data file corresponding to the image-interpretation report based on a content of the image-interpretation report.

### CROSS-REFERENCE TO RELATED APPLICATION

This application is based upon and claims the benefit of priority from Japanese Patent Application No. 2020-147030, filed on Sep. 1, 2020, the entire contents of which are incorporated herein by reference.

### FIELD

An embodiment disclosed in the present specification and drawings relates to a medical data file processing apparatus and a medical data file processing method.

### BACKGROUND

Image data acquired by medical institutions and medical image files (an example of medical data files), which includes medical image data of clinical data such as examination results, samples, and data before analysis acquired in a medical institution, are used for research for developing pharmaceuticals and medical apparatuses. The medical image file can be used by a third party with the consent of the subject (e.g., a patient who has been examined at a medical institution).

The medical image file is provided to the research institution after being given the attribute tag (that is, tag data). As a method of anonymizing tag data, there are a technique of uniformly anonymizing all elements of tag data, a technique of encryption/ decryption, and the like.

### BRIEF DESCRIPTION OF THE DRAWINGS

FIG. 1 is a schematic view showing a configuration of a medical data file processing apparatus according to an embodiment.

FIG. 2 is a schematic view showing a configuration of a medical data file processing system provided with the medical data file processing apparatus according to the embodiment.

FIG. 3 is a block diagram showing an example of functions of the medical data file processing apparatus according to the embodiment.

FIG. 4 is a diagram for explaining a method of determining attribute of the image-interpretation report in the medical data file processing apparatus according to the embodiment.

FIG. 5 is a diagram for explaining a method of determining attribute of the image-interpretation report in the medical data file processing apparatus according to the embodiment.

FIG. 6 is a diagram for explaining a method of classifying medical image file in the medical data file processing apparatus according to the embodiment.

FIG. 7 is a diagram for explaining a method of determining a diagnostic purpose of a medical image file in the medical data file processing apparatus according to the embodiment.

FIG. 8 is a diagram for explaining a method of anonymizing a medical image file in the medical data file processing apparatus according to the embodiment.

FIG. 9 is a diagram showing a method of processing a medical image file as a flowchart in the medical data file processing apparatus according to the embodiment.

FIG. 10 is a diagram for explaining an anonymization method based on a purpose of use of the medical image file.

FIG. 11 is a diagram for explaining an anonymization method based on a purpose of use of the medical image file.

### DETAILED DESCRIPTION

A medical data file processing apparatus and a medical data file processing method according to a present embodiment will be described with reference to the accompanying drawings.

The medical data file processing apparatus according to the present embodiment includes processing circuitry. The processing circuitry is configured to acquire an image-interpretation report, and to classify a medical data file corresponding to the image-interpretation report based on a content of the image-interpretation report.

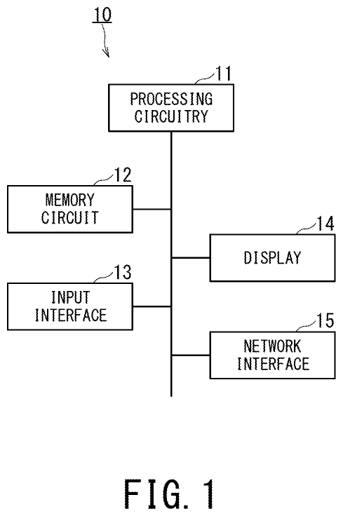

FIG. 1 is a schematic view showing a configuration of a medical data file processing apparatus according to an embodiment.

FIG. 1 shows a medical data file processing apparatus 10 according to an embodiment. The medical data file processing apparatus 10 appropriately anonymizes a medical data file including medical data and attribute tag (that is, tag data) thereof. An example of medical data is medical image data, and an example of a medical data file is a medical image file. Hereinafter, unless otherwise specified, a case where the medical data file processing apparatus 10 appropriately anonymizes a medical image file including medical image data and attribute tag (that is, tag data) thereof will be described. The medical data file processing apparatus 10 is an image-interpretation report managing apparatus (data server), a workstation, an image-interpretation terminal, or the like, and is provided on a medical data system connected via a network. The medical data file processing apparatus 10 may be an offline apparatus.

The medical data file processing apparatus 10 includes processing circuitry 11, a memory circuit 12, an input interface 13, a display 14, and a network interface 15.

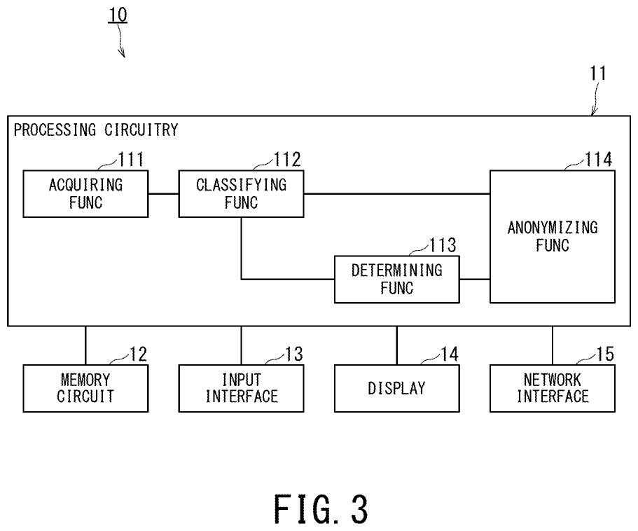

The processing circuitry 11 controls the operation of the medical data file processing apparatus 10 by responding an input operation received from the operator via the input interface 13. For example, the processing circuitry 11 is realized by a processor. The function of the processing circuitry 11 will be described later with reference to FIG. 3.

The memory circuit 12 is constituted by a semiconductor memory element such as a random-access memory (RAM) or a flash memory, a hard disk, an optical disk, or the like. The memory circuit 12 may be constituted by a portable medium such as a universal serial bus (USB) memory and a digital video disk (DVD). The memory circuit 12 stores various processing programs (including an operating system (OS) and the like besides the application program) used in the processing circuitry 11 and data necessary for executing the programs. In addition, the OS may include a graphical user interface (GUI) which may use graphics frequently when displaying data on the display 14 to the operator and can perform basic operations using the input interface 13. The memory circuit 12 is an example of a storage unit.

The input interface 13 includes an input device operable by an operator, and a circuit for inputting a signal from the input device. The input device may be a trackball, a switch, a mouse, a keyboard, a touch pad for performing an input operation by touching an operation surface, a touch screen in which a display screen and a touch pad are integrated, a non-contact input circuit using an optical sensor, an audio input circuit, and the like. When the input device is operated by the operator, the input interface 13 generates an input signal corresponding to the operation and outputs it to the processing circuitry 11. The medical data file processing apparatus 10 may include a touch panel in which the input device is integrally configured with the display 14. Further, the input device is not limited to the one provided with physical operating parts such as a mouse and a keyboard. For example, an example of the input interface 13 includes a configuration in which the input circuit receives an electric signal corresponding to an input operation from an external input device provided separately from the medical data file processing apparatus 10, and the circuit outputs this electric signal to the processing circuitry 11. The input interface 13 is an example of an input unit.

The display 14 may be a liquid crystal display, a cathode ray tube (CRT) display, an organic light emitting diode (OLED) display, or the like. The display 14 is connected to the processing circuitry 11 and displays various data and images generated under the control of the processing circuitry 11. The display 14 is an example of a display unit.

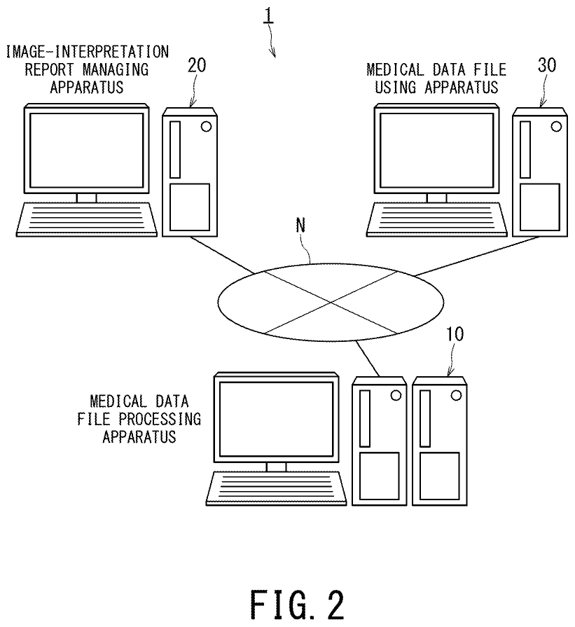

The network interface 15 is configured by composed connectors that meet the parallel connection specifications and the serial connection specifications. The network interface 15 has a function of performing communication control according to each specification and connecting the network N (shown in FIG. 2) through a telephone line. As a result, the network interface 15 can connect the medical data file processing apparatus 10 to the network. The network interface 15 is an example of a network connecting unit.

Subsequently, a system to which the medical data file processing apparatus 10 is applied will be described.

FIG. 2 is a schematic view showing a configuration of a medical data file processing system provided with the medical data file processing apparatus 10.

FIG. 2 shows a medical data file processing system 1 provided with the medical data file processing apparatus 10. The medical data file processing system 1 includes a medical data file processing apparatus 10 shown in FIG. 1, one or more image-interpretation report managing apparatuses (e.g., data servers of medical institutions) 20, and one or more medical data file using apparatuses (e.g., research institute terminals) 30. The medical data file processing apparatus 10, the image-interpretation report managing apparatus 20, and the medical data file using apparatus 30 are connected so as to be able to communicate with each other via the network N. An electrical connection or the like via an electronic network can be applied to this connection. In the present invention, the electronic network refers to a general information communication network using telecommunication technology, which includes a wireless/wired hospital backbone local area network (LAN), an internet network, a telephone communication network, an optical fiber communication network, a cable communication network, a satellite communication network, and the like.

The medical data file processing apparatus 10 is provided inside an anonymizing processing medical institution or outside the anonymizing processing medical institution (e.g., inside the medical institution), and anonymizes the medical data file (e.g., the medical image file) by an appropriate method. For example, the medical data file processing apparatus 10 deletes, changes, or encrypts (or conceals) each element of the tag data of the medical image file.

The image-interpretation report managing apparatus 20 is provided inside the medical institution or outside the medical institution, and manages an image-interpretation report based on the medical image file acquired from an examination or the like of a subject who uses the medical institution. For example, one image-interpretation report managing apparatus 20 is provided for each of the multiple medical institutions. The medical image file includes medical image data and tag data thereof. Examples of medical image data include image data acquired at the medical institution, the image-interpretation report generated based on clinical data such as test results, samples, and pre-analysis data collected at the medical institution. The tag data includes elements of personal information of the subject such as name, age, height, and weight.

The image-interpretation report managing apparatus 20 includes a general configuration of a computer. For example, the image-interpretation report managing apparatus 20 includes processing circuitry, a memory circuit, a DB (Database), an input interface, a display, and a network interface (all not shown). The DB is a storage unit that stores the medical image file with the image-interpretation report. The DB stores medical image data with tag data such as patient information and acquisition date and time under the control of the processing circuitry. Further, the DB provides a specific medical image file according to the request from the stored medical image files under the control of the processing circuitry.

The medical data file using apparatus 30 is a user terminal operated by a researcher who uses medical image data related to the subject. For example, one medical data file using apparatus 30 is provided for each of the multiple research institutes. The medical data file using apparatus 30 may request the medical data file processing apparatus 10 to provide image data of myocardial infarction, image data of hepatocellular carcinoma, or learning data for reconstruction.

The medical data file using apparatus 30 includes a general configuration of a computer, which may include processing circuitry, a memory circuit, an input interface, a display, and a network interface (all not shown).

Subsequently, functions of the medical data file processing apparatus 10 will be described.

FIG. 3 is a block diagram showing an example of functions of the medical data file processing apparatus 10.

As shown in FIG. 3, the processing circuitry 11 reads and executes a computer program stored in the memory circuit 12 or directly embedded in the processing circuitry 11, thereby realizing an acquiring function 111, a classifying function 112, a determining function 113, and an anonymizing function 114. Hereinafter, the case where the functions 111 to 114 realized by execution of the computer program and function as software will be described as an example, but all or a part of the functions 111 to 114 may be realized by a circuit such as an ASIC.

The acquiring function 111 includes a function of acquiring image-interpretation report data via the network N or a portable recording medium. The acquiring function 111 is an example of an acquiring unit.

The classifying function 112 includes a function of analyzing a content of the image-interpretation report acquired by the acquiring function 111, and determining an attribute of the image-interpretation report.

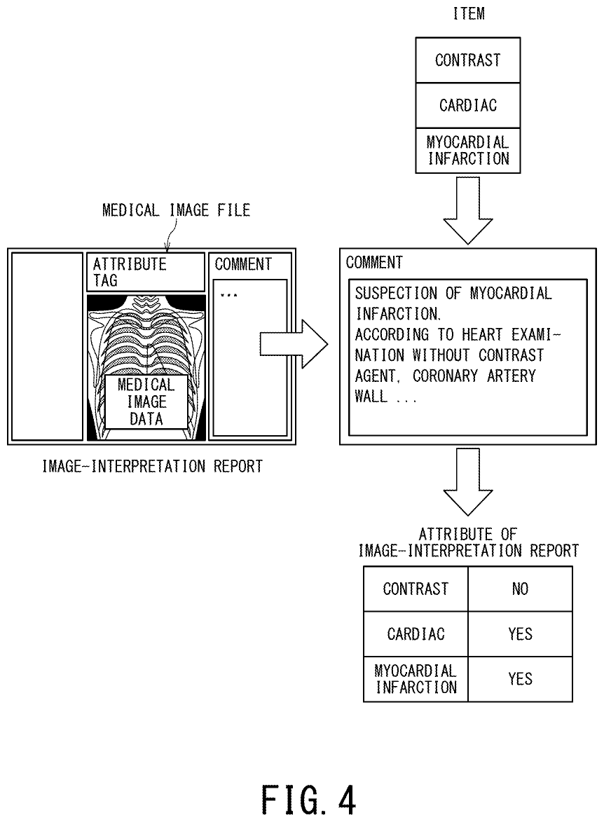

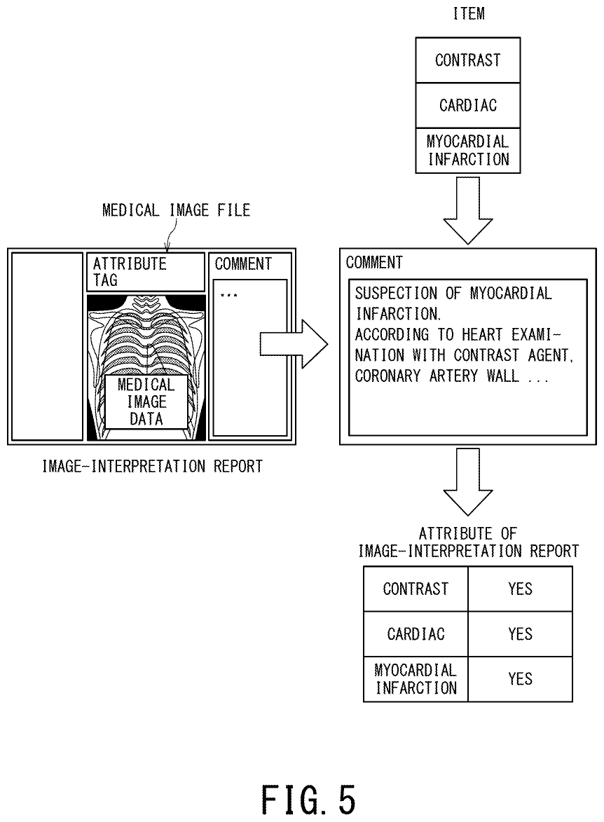

Each of FIGS. 4 and 5 is a diagram for explaining a method of determining the attribute of the image-interpretation report in the medical data file processing apparatus 10.

As shown in FIGS. 4 and 5, the image-interpretation report has a comment field input by an image-interpreter. The classifying function 112 analyzes the comment input by the image-interpreter, and determines whether each preset item is included in the comment (YES) or not included (NO). The classifying function 112 sets a combination of determination result of each item as an attribute of the image-interpretation report. In the example shown in FIG. 4, the attribute of the image-interpretation report is a combination of “contrast: NO”, “cardiac: YES”, and “myocardial infarction: YES”. In the example shown in FIG. 5, the attribute of the image-interpretation report is a combination of “contrast: YES”, “cardiac: YES”, and “myocardial infarction: YES”.

Returning to the description of FIG. 3, the classifying function 112 further includes a function of classifying (or labeling) the medical image file corresponding to the image-interpretation report acquired by the acquiring function 111 (including the medical image data itself and tag data thereof) according to the attribute of the determined image-interpretation report. The classifying function 112 is an example of a classifying unit.

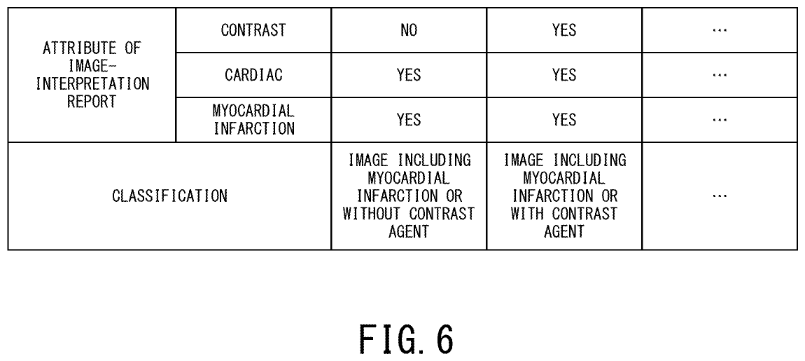

FIG. 6 is a diagram for explaining a method of classifying medical image file in the medical data file processing apparatus 10.

As shown in FIG. 6, classification is set in advance according to the attribute of the image-interpretation report. When the attribute of the image-interpretation report is the combination of “contrast: NO”, “cardiac: YES” and “myocardial infarction: YES” as shown in FIG. 4, the classifying function 112 classifies the medical image file corresponding to the image-interpretation report into “image including myocardial infarction” or “without contrast agent”. “Image including myocardial infarction” corresponds to the major category, and “without contrast agent” corresponds to the minor category.

On the other hand, when the attribute of the image-interpretation report is the combination of “contrast: YES”, “cardiac: YES” and “myocardial infarction: YES” as shown in FIG. 5, the classifying function 112 classifies the medical image file corresponding to the image-interpretation report into “image including myocardial infarction” or “with contrast agent”. “Image including myocardial infarction” corresponds to the major category, and “with contrast agent” corresponds to the minor category.

Returning to the description of FIG. 3, determining function 113 includes a function, based on a content of the image-interpretation report acquired by the acquiring function 111, of determining a diagnostic purpose of medical image file corresponding to the image-interpretation report. The determining function 113 is an example of a determining unit.

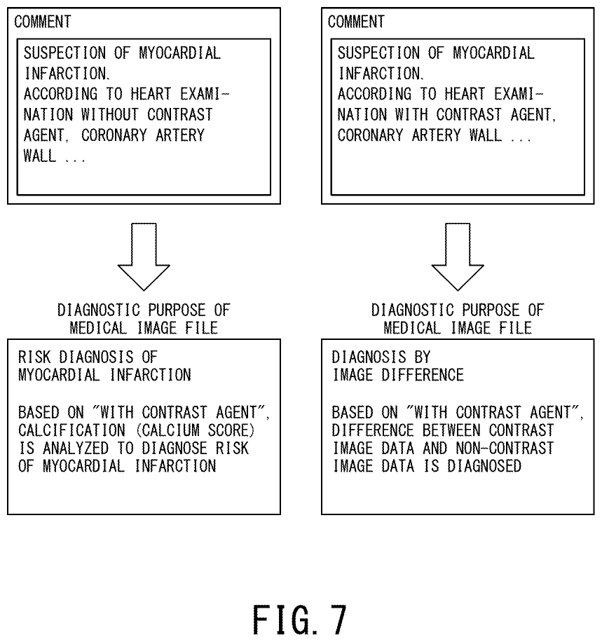

FIG. 7 is a diagram for explaining a method of determining a diagnostic purpose of a medical image file in the medical data file processing apparatus 10.

In the case as the image-interpretation report shows in FIG. 4, the determining function 113 determines a diagnostic purpose of the medical image file as “risk diagnosis of myocardial infarction” on the basis of “without contrast agent”. On the other hand, in the case as the image-interpretation report shows in FIG. 5, the determining function 113 determines a diagnostic purpose of the medical image file as “diagnosis by image difference” on the basis of “with contrast agent”.

In the present invention, the determining function 113 performs a processing of determining the diagnostic purpose of the medical image file based on the comment of the image-interpretation report. For this processing, for example, a look-up table (LUT) that associates the content of the comment with the purpose of diagnosis may be used. In addition, machine learning may be applied to this processing. As machine learning, deep learning using a multi-layer neural network such as CNN (Convolutional Neural Networks), convolutional deep belief network (CDBN), and the like may be used.

Returning to the description of FIG. 3, the anonymizing function 114 includes a function of performing anonymization processing on the basis of a content according to the medical image file corresponding to the image-interpretation report acquired by the acquiring function 111. In that case, the determining function 113 is unnecessary. Alternatively, the anonymizing function 114 includes a function of anonymizing the medical image file corresponding to the image-interpretation report acquired by the acquiring function 111 based on a combination of the classification result by the classifying function 112 and the determination result by the determining function 113. Hereinafter, the case where the anonymizing function 114 performs anonymization processing on the tag data of the medical image file based on the content combing the classification result and the determination result will be described as an example. The anonymizing function 114 is an example of an anonymizing unit.

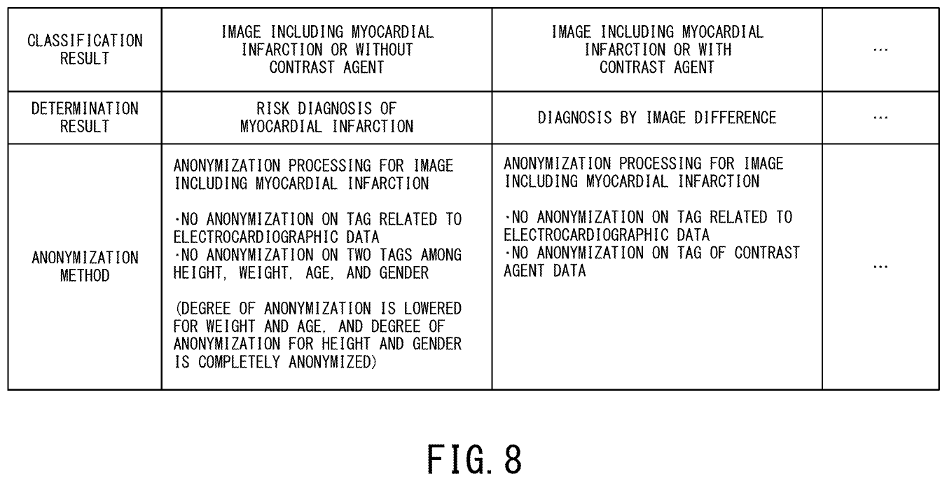

FIG. 8 is a diagram for explaining a method of anonymizing a medical image file in the medical data file processing apparatus 10.

As shown in FIG. 8, when the diagnostic purpose is determined to examine the risk of myocardial infarction, an anonymization method of “No anonymization on the tag related to the electrocardiographic data” and “No anonymization on two tags among height, weight, age, and gender” is assigned. In addition, in that case, anonymization level of weight and age may be lowered, and the height and gender may be completely anonymized. On the other hand, when the diagnostic purpose is determined to examine the difference between the contrast-enhanced image data and the non-contrast-enhanced image data, an anonymization method of “No anonymization on the tag related to electrocardiographic data” and “No anonymization on the tag of the contrast agent data” is assigned. The content of the anonymization processing may include the level (degree) of anonymization. Regarding the level of anonymization, an evaluation index called “k-anonymity” is widely used. By processing each element of the tag data so as to satisfy “k-anonymity”, the probability that an individual is identified can be reduced to 1/k or less.

In this way, the medical data file processing apparatus 10 is possible to classify the medical image file corresponding to the image-interpretation report by analyzing the content of the image-interpretation report. Further, the medical data file processing apparatus 10 determines the diagnostic purpose of the medical image file from the content of the image-interpretation report, thereby determining the anonymization method according to the classification and the diagnostic purpose.

Subsequently, a method of processing the medical image file in the medical data file processing apparatus 10 will be described.

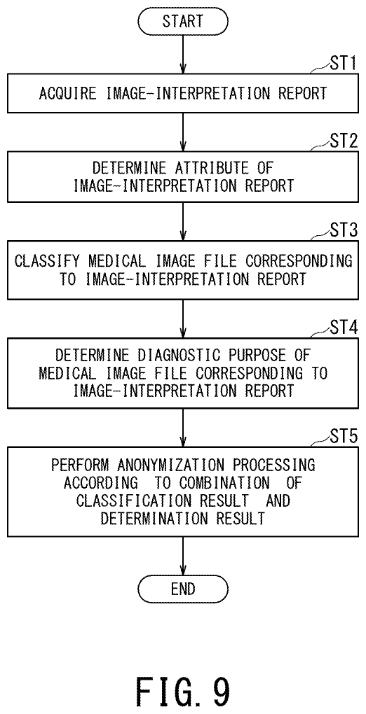

FIG. 9 is a diagram showing a method of processing a medical image file as a flowchart. In FIG. 9, reference numerals with numbers attached to “ST” indicate each step of the flowchart.

The acquiring function 111 acquires data of the image-interpretation report via the network N or the portable recording medium (step ST1). The classifying function 112 analyzes the content of the image-interpretation report acquired by the acquiring function 111 and determines the attribute of the image-interpretation report (step ST2). The method of determining the attribute of the image-interpretation report has been described with reference to FIGS. 4 and 5.

The classifying function 112 classifies the medical image file corresponding to the image-interpretation report (including medical image data itself and tag data thereof) acquired in step ST1 according to the attribute of the image-interpretation report determined in step ST2 (step ST3). The method of classifying the medical image file has been described with reference to FIG. 6. The determining function 113 determines the diagnostic purpose of the medical image file corresponding to the image-interpretation report on the basis of the content of the image-interpretation report acquired in step ST1 (step ST4). The determining of the diagnostic purpose of the medical image file has been described with reference to FIG. 7.

The anonymizing function 114 performs anonymization processing on the medical image file corresponding to the image-interpretation report acquired in step ST1, in accordance with the tag data according to a combination of the classification result in step ST3 and the determination result in step ST4 (step ST5). The anonymization method has been described with reference to FIG. 8.

The medical image file that has been anonymized in step ST5 is provided to the medical data file using apparatus 30 (shown in FIG. 2) via the network N on the request of the medical data file using apparatus 30. In the medical data file using apparatus 30, the anonymization processing includes the level (degree) of anonymization, and the anonymization purpose is not limited to the learning use of AI.

As described above, according to the medical data file processing apparatus 10, not all the data in the medical image file is anonymized. Instead, it is possible to anonymize only the data selected according to the content of the image-interpretation report among the medical image files. As a result, the usage of the medical image file can be more effective and be expanded while anonymizing the data as required.

### First Modified Example

In the above-mentioned example, the case where the method of anonymizing the medical image file is determined based on the diagnostic purpose of the medical image file has been described, but it is not limited to that case. For example, the medical data file processing apparatus 10 may determine a method for anonymizing the medical image file based on a purpose of use of the medical image file in the medical data file using apparatus 30. In that case, the determining function 113 includes a function of determining a purpose of use of the medical image file corresponding to the image-interpretation report in response to a request from the medical data file using apparatus 30.

Further, the anonymizing function 114 includes a function of performing anonymization processing on the medical image file corresponding to the image-interpretation report acquired by the acquiring function 111 on the basis of a content according to a combination of the classification result by the classifying function 112 and the determination result by the determining function 113.

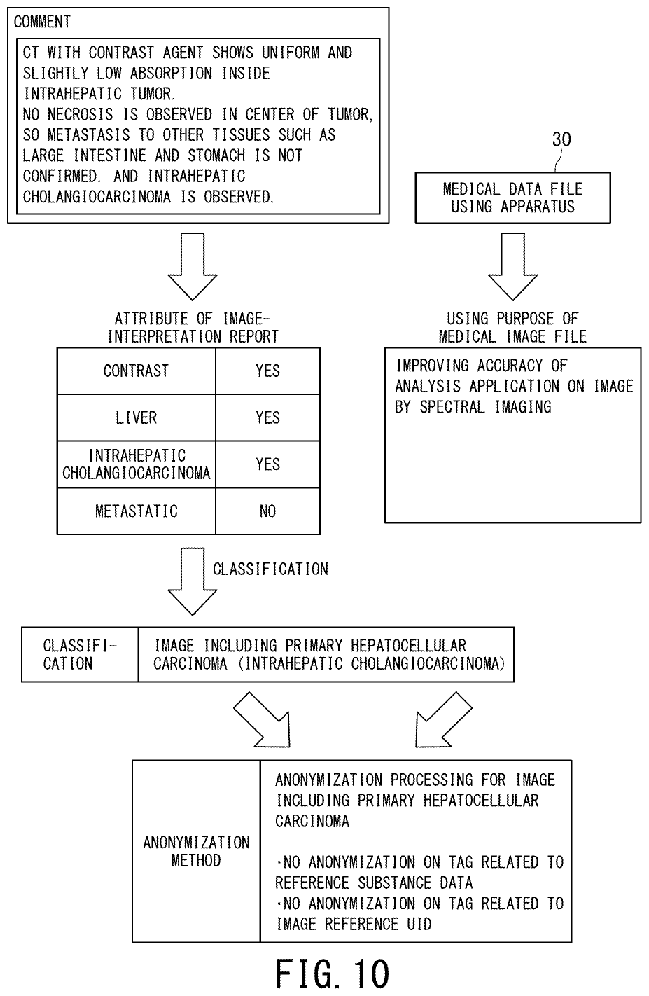

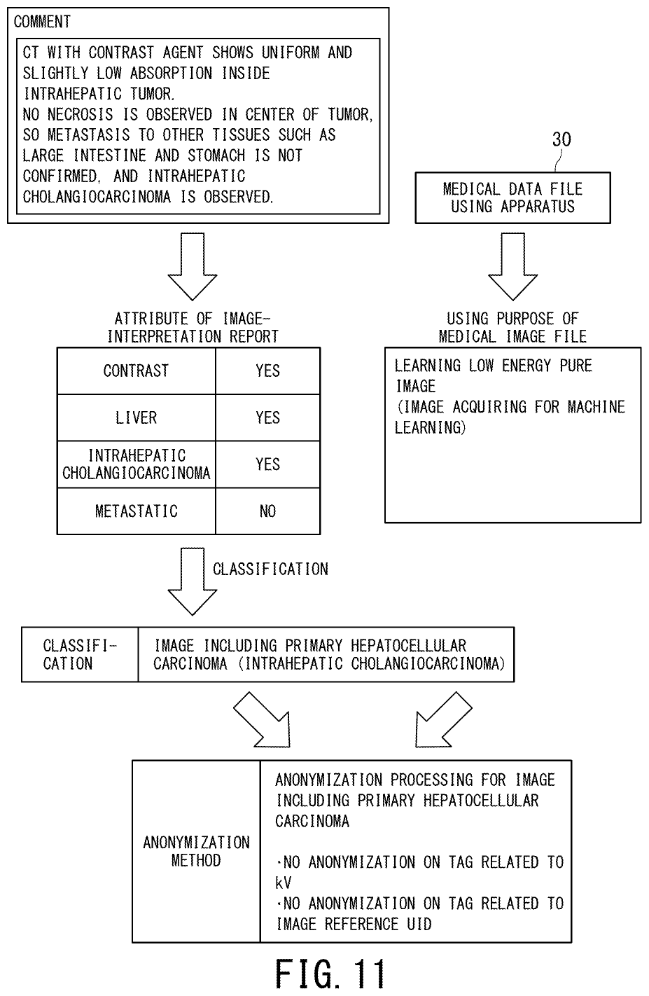

Each of FIGS. 10 and 11 is a diagram for explaining an anonymization method based on the purpose of use of the medical image file.

As shown in FIG. 10, on the use purpose of “improving accuracy of analysis application regarding image by Spectral Imaging”, providing a medical image file is required to be provided by the medical data file using apparatus 30. The anonymizing function 114 determines the anonymization method based on the combination of the purpose of use of “improving accuracy of analysis application regarding image by Spectral Imaging” and the classification result. Since the tag related to reference substance data in the medical image file provided by the medical data file processing apparatus 10 and the tag related to image reference UID are not anonymized, the medical data file using apparatus 30 is able to conduct research including this tag data.

As shown in FIG. 11, on the use purpose of “learning low energy pure image (image that have not been image-processed)”, providing a medical image file is required by the medical data file using apparatus 30. The anonymizing function 114 determines the anonymization method based on the combination of the purpose of use of “learning low energy pure image” and the classification result. Since the tag related to kV (tube voltage) including in the tag data of the medical image file provided by the medical data file processing apparatus 10 and the tag related to image reference UID are not anonymized, the medical data file using apparatus 30 is able to conduct research including this tag data.

In addition to the purpose of use shown in FIGS. 10 and 11, the medical data file using apparatus 30 has a higher-level purpose of use “learning of image including hepatocellular carcinoma image”, and lower-level purposes of use including “improvement in accuracy of calcium score” and “improvement in accuracy of subtraction between non-contrast image data and contrast image data”. Further, the medical data file using apparatus 30 has a higher-level purpose of use of “learning of Spectral Imaging”, and lower-level purposes of use including “improvement in accuracy of analysis application for Spectral Imaging” and “reconstruction of low energy pure image with high definition”. The anonymizing function 114 is able to determine the anonymization method according to the purpose of use of the higher or lower level in the medical data file using apparatus 30.

In the present invention, the determining function 113 performs a processing of determining the purpose of use of the medical image file based on the request content of the medical data file using apparatus 30. For this processing, for example, a LUT that associates the request content with the purpose of use may be used. In addition, machine learning may be applied to this processing. Further, as machine learning, deep learning using a multi-layer neural network such as CNN or a convolutional deep belief network may be used.

As described above, according to the medical data file processing apparatus 10 (including the first modified example), not all the data of the medical image file (an example of the medical data file) is anonymized. It is possible to anonymize only the data selected according to the content of the image-interpretation report and the request from the researcher among the medical image files. As a result, the usage of the medical image file can be more effective and be expanded while anonymizing the data t as required.

### Second Modified Example

The case where the medical data file processing apparatus 10 classifies the medical image file (an example of the medical data file) corresponding to the image-interpretation report on the basis of the content of the image-interpretation report has been described above. However, it is not limited to that case. For example, the medical data file processing apparatus 10 may be configured to classify a medical data file other than the medical image file (e.g., a medical record data file including medical record data) on the basis of the content of the image-interpretation report. In the present invention, the medical record data file is stored in a system (not shown) in the medical data file processing system 1, and refers to a file including all kinds of data related to a patient, such as a patient's individual health record and medical history. For example, the system includes a hospital information system (HIS) and a radiological information system (RIS).

In the second modified example of the medical data file processing apparatus 10, a computer program (anonymization application), which is provided in the medical data file processing apparatus 10 or in the medical data file processing system 1, is used to appropriately anonymize the medical record data file including the medical record data and tag data thereof.

The configurations, functions, and operations of the second modified example of the medical data file processing apparatus 10 (including the first modified example) are the same as those of the medical data file processing apparatus 10 described above with reference to FIGS. 1 to 11. Therefore, those explanations will be omitted.

As described above, according to the second modified example of the medical data file processing apparatus 10, not all the data of the medical record data file (an example of the medical data file) is anonymized. It is possible to anonymize only the data selected according to the content of the image-interpretation report and the request from the researcher among the medical record data files. As a result, the usage of the medical record data file can be more effective and be expanded while anonymizing the data as required.

The medical data file processing apparatus 10 is not limited to the case where the classification is performed based on the content of the image-interpretation report. The medical data file processing apparatus 10 may classify the medical data file based on a content of medical record data.

According to at least one embodiment described above, it is possible to classify the medical data file according to the content of the corresponding image-interpretation report.

While certain embodiments have been described, these embodiments have been presented by way of example only, and are not intended to limit the scope of the inventions. Indeed, the novel methods and systems described herein may be embodied in a variety of other forms; furthermore, various omissions, substitutions, changes, and combinations of embodiments in the form of the methods and systems described herein may be made without departing from the spirit of the inventions. The accompanying claims and their equivalents are intended to cover such forms or modifications as would fall within the scope and spirit of the inventions.

What is claimed is:

1. A medical data file processing apparatus comprising: processing circuitry configured to
acquire an image-interpretation report; and
classify a medical data file corresponding to the image-interpretation report based on a content of the image-interpretation report.

2. The medical data file processing apparatus according to claim 1, wherein
the processing circuitry is configured to perform anonymization processing on the medical data file based on a content according to the classification result.

3. The medical data file processing apparatus according to claim 1, wherein
the processing circuitry is configured to
determine a diagnostic purpose of the medical data file corresponding to the image-interpretation report based on the content of the image-interpretation report; and
perform anonymization processing on the medical data file based on a content according to a combination of the classification result and the determination result.

4. The medical data file processing apparatus according to claim 1, wherein
the processing circuitry is configured to
determine a purpose of use of the medical data file; and
perform anonymization processing on the medical data file based on a content according to a combination of the classification result and the determination result.

5. The medical data file processing apparatus according to claim 1, wherein
the medical data file includes medical data and attribute tag thereof.

6. The medical data file processing apparatus according to claim 2, wherein
the medical data file includes medical data and attribute tag thereof.

7. The medical data file processing apparatus according to claim 3, wherein
the medical data file includes medical data and attribute tag thereof.

8. The medical data file processing apparatus according to claim 4, wherein
the medical data file includes medical data and attribute tag thereof.

9. The medical data file processing apparatus according to claim 1, wherein
the medical data file is a medical image file including medical image data.

10. The medical data file processing apparatus according to claim 1, wherein
the medical data file is a medical record data file including medical record data.

11. A medical data file processing method comprising: steps of
acquiring an image-interpretation report; and
classifying a medical data file corresponding to the image-interpretation report based on a content of the image-interpretation report.
